(19) United States
(12) Patent Application Publication
(10) Pub. No.: US 2003/0119176 A1
(43) Pub. Date: Jun. 26, 2003

(54) Microfabricated instrument for tissue biopsy and genetic analysis

(75) Inventors: Peter Krulevitch, Pleasanton, CA (US); Julie Hamilton, Tracy, CA (US); William J. Benett, Livermore, CA (US)

Correspondence Address: Alan H. Thompson, Assistant Laboratory Counsel, Lawrence Livermore National Laboratory, P.O. Box 808, L-703, Livermore, CA, 94551, US

(73) Assignee: The Regents of the University of California

(21) Appl. No.: 10/032,257

(22) Filed: Dec. 21, 2001

(51) Int. Cl. C12M 1/34

(52) U.S. Cl. 435/287.2; 435/288.5; 435/288.7

(57) ABSTRACT

A microfabricated instrument for tissue biopsy and genetic analysis. The instrument is specifically designed to take tissue and blood samples and carry out genetic (DNA) assays in an integrated reaction chamber. The instrument is portable and has the ability to take small tissue or blood samples in a minimally invasive manner from localized regions, then immediately perform the DNA analysis through a technique such as real-time PCR. Thus, this instrument links a minimally invasive micro-biopsy tool with microfluidic channels and a PCR reaction chamber into a single disposable device. The instrument can be used to rapidly detect and identify people or the presence of disease, with applications in both the military and civilian sectors

The United States Government has rights in this invention pursuant to Contract No. W-7405-ENG-48 between the United States Department of Energy and the University of California for the operation of Lawrence Livermore National Laboratory.

### BACKGROUND OF THE INVENTION

The present invention relates to preparing tissue samples for pathological studies, particularly to an instrument for tissue biopsy and analysis, and more particularly to a microfabricated minimally invasive instrument for taking tissue and blood samples and which includes microfluidic channel and a polymerise chain reaction (PCR) reaction chamber for deoxyribonucleic acid (DNA) analysis, all in a single disposable device.

Genetic analysis is now a commonly used technique for detecting the presence and potential for disease. Blood samples can be used for the DNA analysis, or tissue samples can be taken. The ability to take small tissue or blood samples in a minimally-invasive manner for localized regions, then immediately perform the DNA analysis through a technique such as real-time PCR with a portable instrument would be extremely valuable. Currently, a tissue or blood sample is taken, then delivered to a small container such as a cuvette, then appropriate chemicals are added for cell lysis. Finally, the DNA is extracted and placed in a chamber with a PCR “cocktail” and the PCR analysis is run.

Recently, a microfabricated biopsy/histology instrument has been developed using a combined tissue cutter and specimen treatment chamber, which minimizes specimen handling, but did not include a PCR reaction chamber to immediately perform the DNA analysis. That microfabricated biopsy/histology instrument is described and claimed in U.S. Pat. No. 5,985,217, issued Nov. 16, 1999 to P. A. Krulevitch et al.

The present invention provides a solution to the need for a portable instrument that can carry out sample taking and real-time PCR analysis. The instrument of the present invention provides linking a minimally invasive microbiopsy tool with specimen treatment microfluidic channels and a PCR reaction chamber in a single disposable device. The chip (device) can be used with a handheld PCR instrument to enable genetic analysis in the field. This instrument could be used to rapidly detect and identify people or the presence of disease with applications in both military and civilian sectors.

The present invention constitutes and improvement over the system of above referenced U.S. Pat. No. 5,985,217 by combining a minimally invasive biopsy sampling tool, microfluidic channels for treatment of a sample, and a PCR reaction chamber mounted to or integral with the microfluidic channel.

### SUMMARY OF THE INVENTION

It is an object of the present invention to provide a microfabricated instrument for tissue biopsy and PCR analysis.

A further object of the invention is to provide a portable microfabricated instrument for carrying out real-time DNA analysis.

Another object of the invention is to provide a single instrument for carrying out minimally invasive cutting of sample tissue combined with microchannels for treatment of the tissue sample, and with a PCR reaction chamber for performing DNA analysis.

Another object of the invention is to provide an instrument having microfluidic channels connected at one end to a minimally invasive biopsy tool and at an opposite end with a PCR reaction chamber than can be integral with the microfluidic channels, or constructed to abut the channels for receiving sample therefrom.

Other objects and advantages of the present invention will become apparent from the following description and accompanying drawings. The present invention involves a microfabricated instrument for tissue biopsy and analysis. The instrument of this invention provides minimal specimen handling for carrying out DNA analysis in a PCR reaction chamber. The instrument includes a biopsy section having smooth cutting edges with atomic sharpness capable of slicing or scraping very thin specimens (about 2 μm or greater), a specimen treatment microchannel section involving microliter volumes of chemicals for treating the specimens, and a PCR reaction chamber section for carrying out DNA analysis. The biopsy section of the instrument may be constructed of silicon, ceramics, metals, or other hard materials to enable the forming of the sharp edge cutter, such as by anisotropic etching of silicon and the microchannel and PCR sections may be constructed of silicon, glass, or suitable plastic materials. For example, the device could be made from a very small stainless steel tube having a sharp notch cut into it. When dragged against tissue (such as inside a blood vessel) cells will be scraped off by the notch and captured inside the tube. Then the cells could be washed out of the tube into a PCR chamber that is the tube itself forms a “microchannel” of sorts. The instrument may be constructed to include the three sections on a single chip or substrate or with the PCR reaction chamber section on a separate chip constructed to align with and abut to the microchannel section.

### BRIEF DESCRIPTION OF THE DRAWINGS

The accompanying drawings, which are incorporated into and form a part of the disclosure, illustrate embodiments of the invention and, together with the description, serve to explain the principles of the invention.

FIG. 1 is a partial cross-sectional view of a prior art embodiment of a silicon micromachined tissue cutter and connection microchannels for carrying specimen analysis.

FIG. 2 is a cross-section of an embodiment of an instrument made in accordance with the present invention having an integrated biopsy section, a microchannel specimen treatment section, and a PCR reaction chamber analysis section.

FIG. 3 is a top view of an instrument similar to FIG. 2 showing an inlet microchannel connected to supply chemicals to the microchannel section of the system, and an outlet channel connected to the PCR reaction chamber.

FIG. 4 illustrates an embodiment similar to that of FIG. 3 except for a narrow biopsy section and microchannel section.

FIG. 5 illustrates an embodiment similar to FIG. 4 except the PCR analysis section is separate from, but abutted to the microchannel section.

### DETAILED DESCRIPTION OF THE INVENTION

The present invention involves an instrument for tissue biopsy and genetic analysis. The present invention provides the ability to take small tissue or blood samples in a minimally invasive manner from localized regions, then immediately perform DNA analysis through a real-time PCR with a portable instrument. The present invention links the minimally invasive microbiopsy tool with specimen treatment microfluidic channels and a PCR reaction chamber into a single disposable device. Another approach is to have the microbiopsy chip simple drops samples into a unit containing microchannels and has PCR capability. The key is that this invention minimizes sample handling and enables very small, selective samples to be taken. Other features include sorting of cells so that the PCR could be performed on very specific cells from the overall sample, or filtering to select specific cells. The PCR reaction chamber may be formed integral with or formed separately from the microfluidic channel and microbiopsy tool.

The present invention utilizes a microfabricated instrument for tissue biopsy and sample treatment in a microchannel arrangement similar to that of the above referenced U.S. Pat. No. 5,985,217, and the details of the biopsy tool set forth therein are incorporated herein by reference. The present invention adds to the instrument of U.S. Pat. No. 5,985,217 a PCR reaction chamber, which enables real-time DNA analysis of a sample, cut from tissue by the biopsy section and treated in a microchannel section of the instrument. The instrument also integrates channels for introducing fluids into the microchannel section. While only one inlet channel is shown, additional inlet channels can be utilized for introducing cell lysing solution, or other sample treatment solutions, to lyse or otherwise treat the biopsied tissue. The PCR reaction chamber can be an integrated section of the overall instrument to provide for DNA analysis, or another option is to provide a separate PCR chamber that docks or abuts with the microchannel section in order to transfer the lysed or unlysed biopsied sample to the PCR chamber. The instrument involves a silicon/glass chip, which could be disposable and would both take the biopsy sample and serve as a specimen cartridge for linking to a microfluidic system for introducing chemicals and transporting the sample. As shown in one embodiment the biopsy section and microchannel section can be constructed to define a narrow tip to ensure it is minimally invasive. If desired, the PCR chamber section and the biopsy/microchannel sections may be separately located, and a pipette or other means of transferring the fluid/sample could be used.

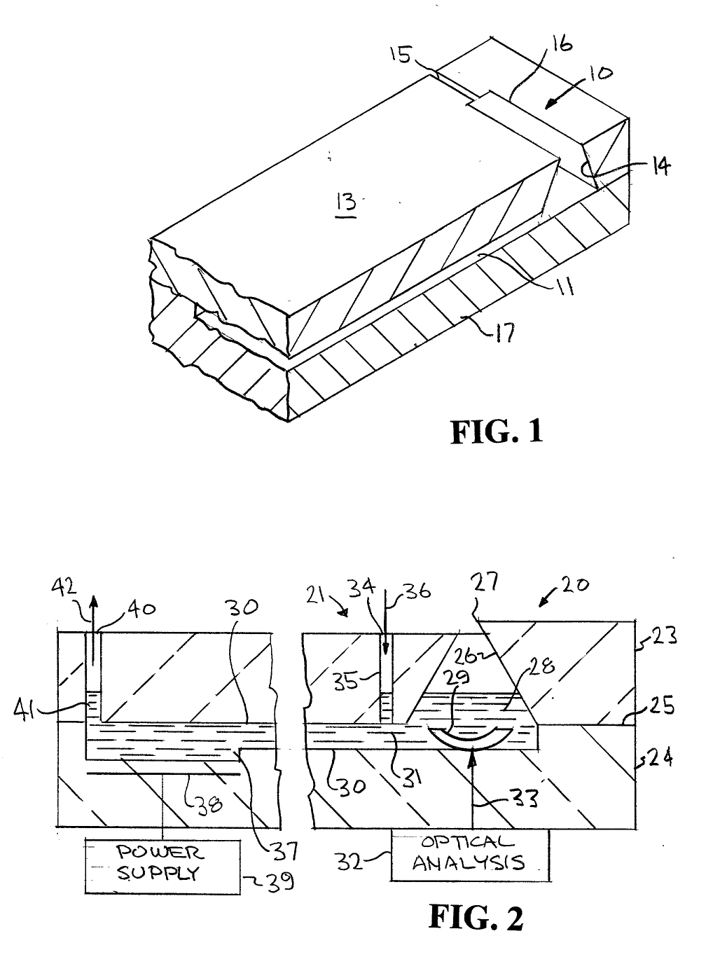

Referring now to the drawings, FIG. 1 illustrates a partial, cross-sectional embodiment of a biopsy/microchannel device, constructed in accordance with above-referenced U.S. Pat. No. 5,985,217, and comprises a cutter 10, a microchannel 11 connected to cutter 10 via a sample or specimen chamber or collection pit 12. The cutter 10 is composed of a silicon substrate or body 13 having a tapered opening or slot 14 with a tapered trailing edge 15 which is located adjacent to a sharp leading edge 16 of tapered slot 14. The depth of the trailing edge 15 may be designed for cutting specimens 2-15 μm thick for example. The substrate or body 13 is composed of silicon and the slot 14 and the sharp edge 16 are etched therein as described above. The substrate 13 or body 13 is bonded to a glass substrate or member 17, which is provided with a plurality of microchannels 11, only one showing whereby the tapered slot 14 terminates adjacent the microchannel to it.

FIG. 2 illustrates an embodiment of an instrument of the invention which incorporates a cutter section 20 and a microchannel section 21, as in FIG. 1, and additionally incorporates a PCR reaction chamber section 22. The instrument is composed of two substrates or members, a silicon substrate 23 and a glass substrate 24 bonded together as indicated at 25. The cutter section 20 is constructed as described in FIG. 1 with a tapered opening or slot 26 having a sharp edge 27 and forming with microchannel section 21 a specimen collection chamber or pit 28, in which is located a cut tissue sample or specimen 29.

The specimen treatment microchannel section 21 includes any desired number of channels 30 formed in glass substrate 24, only one being shown, and which contains a chemical solution 31 for treatment of the tissue specimen or sample 29 Substrate 24 may be made of transparent quartz or plastic. Optical analysis of the sample as it moves along channel 30 is accomplished as indicated at 32 and arrow 33 by a conventionally known optical detector. The chemical solution 31 is supplied to channel 30 via an inlet opening 34 and microchannel 35 formed in silicon member 23, as indicated by arrow 36. A window/optical access for excitation light may be added to the device.

The PCR reaction chamber section 22 is formed in glass member or substrate 24 and includes a reaction chamber 37 connected to receive sample via microchannel 30. The PCR section 22 includes a heater 38 controlled and powered by a power supply 39, and an outlet port 40 connected to chamber 37 via a microchannel 41 for discharge from the chamber 37 as indicated by arrow 42. The operation and function of a PCR reaction chamber is known and thus need not be described.

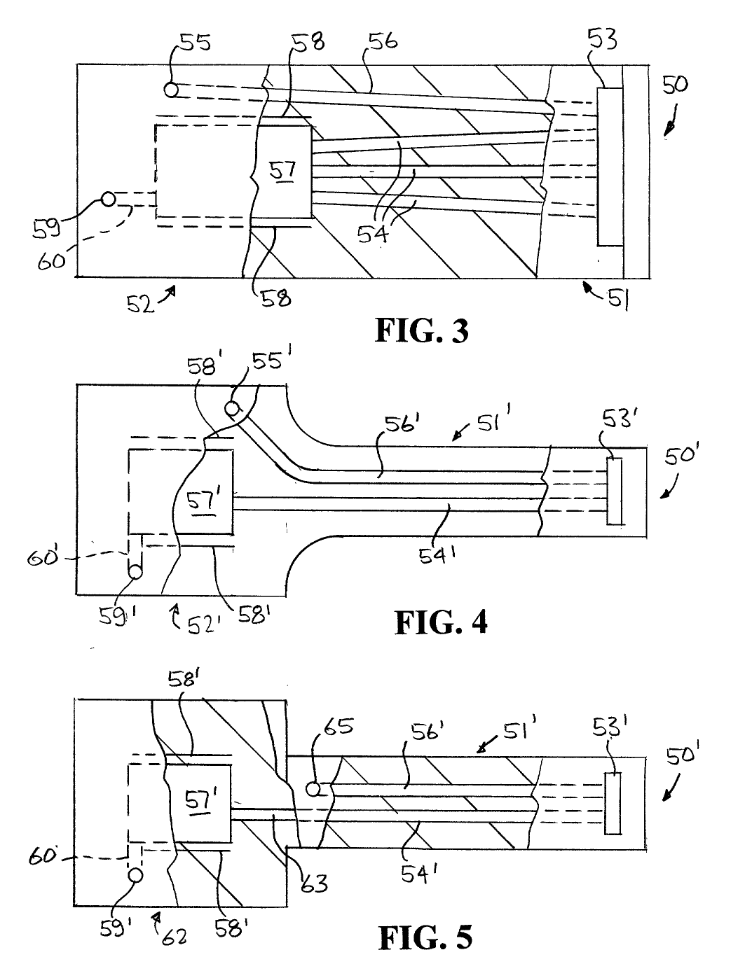

FIGS. 3, 4 and 5 are top views of three different embodiments of a microfabricated instrument for tissue biopsy and genetic analysis, made in accordance with the present invention and include a cutter or biopsy section, a microchannel section and a PCR chamber section, similar to the embodiment of FIG. 2. The instrument of FIG. 3 comprises a cutter section 50, a microchannel section 51 and a PCR chamber section 52. Cutter section 50 includes a cutter 53, constructed as in FIGS. 1 and 2. Microchannel section 52 includes a plurality of microchannels 54 interconnecting cutter section 50 with PCR chamber section 52, and includes an inlet 55 and microchannel 56 which terminates at cutter section 50 and supplies an appropriate chemical solution to microchannels 54 for processing a sample or specimen as described above. PCR chamber section 52 includes a reaction chamber 57, heaters 58, and an outlet port 59 with connecting microchannel 60. As pointed out above, a sample cut by cutter 53 is processed as it passes along a microchannel 54 into the PCR reaction chamber 57 wherein DNA analysis is carried out, as known in the art. As pointed out above, additional inlets and associated microchannels may be added if additional chemical solutions are needed for the process.

The embodiment of FIG. 4 differs from that of FIG. 3 primarily by incorporating a narrow cutter section and microchannel section to proform minimally invasive tissue biopsy. The components of FIG. 4 are the same as in FIG. 3 except for the number of processing microchannels, with the FIG. 4 embodiment illustrating only one, and thus corresponding components are given. With cutter section 50¹ and microchannel section 51¹ being narrow, having a width of 10 microns to 1 mm, tissue samples can be taken in a minimally invasive manner.

The embodiment of FIG. 5 differs from that of FIG. 4 in that the PCR chamber section is not integral with the microchannel and cutter sections, but is designed to mate directly with the microchannel section, as shown. However, use of a pipette or other means of transferring the fluid/sample from the microchannel section to the PCR chamber section can effectively utilize the instrument. Also, the PCR chamber may be a conventional sized PCR instrument, as well as, a micro/mini sized unit. Components corresponding to those of FIG. 4 are given like reference numerals. As shown the integral cutter section 50¹ and microchannel section 51¹ are abutted to or mated with a separate (non-integrated) PCR chamber section 62 having a microchannel 63 extending from reaction chamber 57¹ and aligned with microchannel 54¹ of microchannel section 51¹. The other components of PCR chamber section 61 are the same as in the FIG. 4 embodiment. Microchannel section 51¹ of FIG. 5 has been modified, compared to FIG. 4, by placing the chemical solution inlet 65 for microchannel 56¹ in the microchannel section 51¹.

It has been shown that the present invention provides a portable instrument that has the ability to take small tissue or blood samples in a minimally invasive manner from localized regions, then immediately perform the DNA analysis through real-time PCR. The PCR chamber can be integral with the biopsy and microchannel sections of the instrument, or be on a separate substrate with the capability to mate with the microchannel and biopsy substrate.

This instrument enables genetic analysis in the field by providing a hand held device, and the instrument can be used to rapidly detect and identify people or the presence of disease, with applications in both military and civilian sectors. The instrument provides the capability for biopsy and genetic analysis of tissue and blood cells, for the study of existing or potential medical disorders.

While particular embodiments have been illustrated and described to exemplify and teach the principles of the invention, such are not intended to be limiting. Modifications and changes may become apparent to those skilled in the art and it is intended that the invention be limited only by the scope of the appended claims

The invention claimed is:

1. A microfabricated biopsy/genetic analysis instrument, comprising:
a cutter section,
a specimen chamber located adjacent said cutter section,
a specimen treatment section located adjacent said specimen chamber, and
a PCR reaction chamber section located adjacent said specimen treatment section.

2. The instrument of claim 1, wherein said cutter section includes a member having an opening therein having a smooth cutting edge with atomic sharpness.

3. The instrument of claim 2, wherein said member is constructed of material selected from the group consisting of silicon, metal, ceramic, and other hard material.

4. The instrument of claim 2, wherein said opening has a tapered configuration located adjacent said specimen chamber.

5. The instrument of claim 1, wherein said specimen treatment section includes another member bonded to said first mentioned member and having a plurality of microchannels therein, at least one of said microchannels interconnecting said specimen chamber with said PCR reaction chamber section.

6. The instrument of claim 5, wherein said another member is constructed of material selected from the group consisting of glass, quartz, plastic, and other transparent material.

7. The instrument of claim 5, additionally including at least one fluid inlet and at least one microchannel connected thereto for supplying a fluid to at least said specimen chamber and said at least one of said plurality of microchannels in said specimen treatment section.

8. The instrument of claim 5, additionally including an optical viewing and/or optical excitation arrangement adjacent said specimen treatment section.

9. The instrument of claim 5, wherein said PCR reaction chamber is located in said another member, and includes at least one heater located in said another member.

10. The instrument of claim 9, additionally including at least one outlet and at least one microchannel connected to said PCR reaction chamber.

11. The instrument of claim 1, wherein said PCR reaction chamber section has a cross section greater than a cross section of said specimen treatment section.

12. The instrument of claim 1, wherein said cutter section and said specimen treatment section has a width less than a width of said PCR reaction chamber section.

13. The instrument of claim 12, wherein said PCR reaction chamber section is formed on a separate member than said specimen treatment section and is constructed to abut and align with said specimen treatment section such that fluid/sample passing through said specimen treatment section is directed into said PCR reaction chamber section.

14. The instrument of claim 1, wherein said cutter section, said specimen chamber, and said specimen treatment section are located on one substrate, and wherein said PCR reaction chamber section is located on another substrate, constructed to abut with and align with said specimen treatment section to receive fluid/sample from said specimen treatment section.

15. The instrument of claim 14, wherein said cutter section is located in a member bonded to said one substrate and said specimen treating section is formed in said one substrate.

16. In a biopsy/analysis instrument having a cutter section and a specimen treatment section, the improvement comprising:
a PCR reaction chamber section constructed to receive fluid/sample from said specimen treatment section.

17. The improvement of claim 16, wherein said PCR reaction chamber section, said specimen treatment section, and said cutter section are located on a single substrate.

18. The improvement of claim 16, wherein said cutter section and said specimen treatment section have a width less than a width of said PCR reaction chamber section.

19. The improvement of claim 16, additionally including an optical detection system located adjacent said specimen treatment section.
